(19) United States
(12) Patent Application Publication
(10) Pub. No.: US 2024/0371531 A1
(43) Pub. Date: Nov. 7, 2024

(54) SYSTEM AND METHOD FOR DYNAMIC INTERACTION TO MEDICAL QUERIES PROPOSITIONED CONTEMPORANEOUSLY WITH INTERACTION WITH MEDICAL PERSONNEL

(71) Applicant: Kate R. Lyon, Des Moines, IA (US)

(72) Inventor: Kate R. Lyon, Des Moines, IA (US)

(21) Appl. No.: 18/654,694

(22) Filed: May 3, 2024

Related U.S. Application Data
(60) Provisional application No. 63/500,073, filed on May 4, 2023.

(51) Int. Cl. G16H 80/00; G16H 40/20

(52) U.S. Cl. CPC G16H 80/00; G16H 40/20

(57) ABSTRACT

A system and method for dynamic interaction to medical queries propositioned contemporaneously with interaction with medical personnel that includes at least one wireless device for inputting at least one query regarding a medical condition, a computer network in electronic communication with the at least one wireless device, and at least one processor that utilizes a large language model (LLM) that is in electronic communication with the computer network for analyzing the at least one query from the at least one wireless device and provide at least one follow up query to the at least one wireless device to generate at least one additional input from the at least one wireless device to provide a medical information contemporaneously with communication with medical personnel. Medical information can include a medical diagnosis or a restatement of a question for medical personnel. In addition, medical data from a wearable sensor may be provided.

### CROSS REFERENCE TO RELATED APPLICATIONS

This application claims priority under 35 U.S.C. § 119 to provisional patent application U.S. Ser. No. 63/500,073, filed May 4, 2023. The provisional patent application is herein incorporated by reference in its entirety, including without limitation, the specification, claims, and abstract, as well as any figures, tables, appendices, or drawings thereof.

### TECHNICAL FIELD

The present disclosure generally relates to a system and method for dynamic interaction to medical queries propositioned contemporaneously with interaction with medical personnel.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Currently, patients can be frustrated by medical providers who do not sufficiently explain the medical issue to the patient during a visit. As a result, the patient typically lacks recourse and does not have enough information to make the best decision regarding treatment. In the alternative, questions can be posed to a static website that does not provide enough detail to address the specifics of the patient's condition. There is a tremendous need to generate medical analysis contemporaneously with receiving information from medical personnel in order to optimize healthcare treatment.

### SUMMARY

The following objects, features, advantages, aspects, and/or embodiments, are not exhaustive and do not limit the overall disclosure. No single embodiment needs to provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

An aspect of the present invention is a system for dynamic interaction to medical queries propositioned contemporaneously with interaction with medical personnel. The system includes at least one wireless device for inputting at least one query regarding a medical condition, a computer network in electronic communication with the at least one wireless device, and at least one processor that utilizes a large language model (LLM) that is in electronic communication with the computer network for analyzing the at least one query from the at least one wireless device and provide at least one follow up query to the at least one wireless device to generate at least one additional input from the at least one wireless device to provide medical information that includes, but is not limited to, a medical diagnosis or a restatement of a query for medical personnel, contemporaneously with communication with medical personnel.

Another aspect of the present invention is at least one wireless device, and at least one processor that utilizes a large language model (LLM) exchanges a plurality of queries through the computer network prior to the at least one processor arriving at medical information that includes, but is not limited to, a medical diagnosis or a restatement of a query for medical personnel,

Still, another aspect of the present invention is the wireless device includes at least one cell phone.

Still, yet another aspect of the present invention is the wireless device includes at least one electronic tablet.

Another aspect of the present invention is the wireless device includes at least one wearable sensor.

In yet another aspect of the present invention, at least one wearable sensor by a patient provides additional medical data that is in electronic communication with the at least one wireless device.

In another aspect of the present invention is a wearable sensor selected from the group consisting of a heart sensor, an eye sensor, a respiratory sensor, a skin sensor, an activity sensor, a sleep sensor, an ear health sensor, a cognitive function sensor, and a mental health sensor.

Still another aspect of the present invention is a system for dynamic interaction to medical queries propositioned contemporaneously with interaction with medical personnel, the system includes at least one wireless device for inputting at least one query regarding a medical condition, a computer network in electronic communication with the at least one wireless device, at least one wearable sensor by a patient for providing additional medical data that is in electronic communication with the at least one wireless device, and at least one processor that utilizes a large language model (LLM) that is in electronic communication with the computer network for analyzing the at least one query from the at least one wireless device and provide at least one follow up query to the at least one wireless device to generate at least one additional input from the at least one wireless device combined with the additional medical data from the at least one wearable sensor to provide a medical information that includes, but is not limited to, a medical diagnosis or a restatement of a query for medical personnel, contemporaneously with communication with medical personnel.

An additional aspect of the present invention is the at least one wireless device is selected from the group consisting of at least one cell phone, at least one electronic tablet, and at least one wearable electronic device.

In another aspect of the present invention is a process for dynamic interaction to medical queries propositioned contemporaneously with interaction with medical personnel, the process includes inputting a query into at least one wireless device regarding a medical condition, wherein the at least one wireless device is in electronic communication with a computer network, wherein the computer network is in electronic communication with at least one processor that utilizes a large language model (LLM), analyzing the at least one query from the at least one wireless device with the least one processor that utilizes a large language model (LLM), providing at least one follow up query to the at least one wireless device from the least one processor that utilizes a large language model (LLM), receiving at least one additional input from the at least one wireless device, and providing a medical information that includes, but is not limited, to a medical diagnosis or a restatement of a query for medical personnel, to the at least one wireless device from the at least one processor that utilizes a large language model (LLM) contemporaneously with interaction with medical personnel.

Still, another aspect of the present invention is obtaining additional medical data through at least one wearable sensor by a patient that is in electronic communication with the at least one wireless device.

An additional aspect of the present invention is the step of combining the additional medical data from a wearable sensor with the inputted response to at least one query regarding a medical condition to obtain medical information that includes, but is not limited to, a medical diagnosis or a restatement of a query for medical personnel, with the at least one processor that utilizes a large language model (LLM) contemporaneously with interaction with medical personnel.

In another aspect of the present invention is the step of inputting a query into at least one wireless device, which includes a patient typing input.

Yet another aspect of the present invention is the step of inputting a query into at least one wireless device, which includes voice recognition of a patient.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 is a block diagram showing a computer system configured to implement the system of the present invention for dynamic interaction to medical queries propositioned contemporaneously with interaction with medical personnel.

FIG. 2 is a flowchart of the process steps associated with the present invention for dynamic interaction to medical queries propositioned contemporaneously with interaction with medical personnel.

An artisan of ordinary skill in the art need not view, within the isolated figure(s), the near-infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. Unless otherwise indicated, no features shown or described are essential to permit basic operation of the present disclosure.

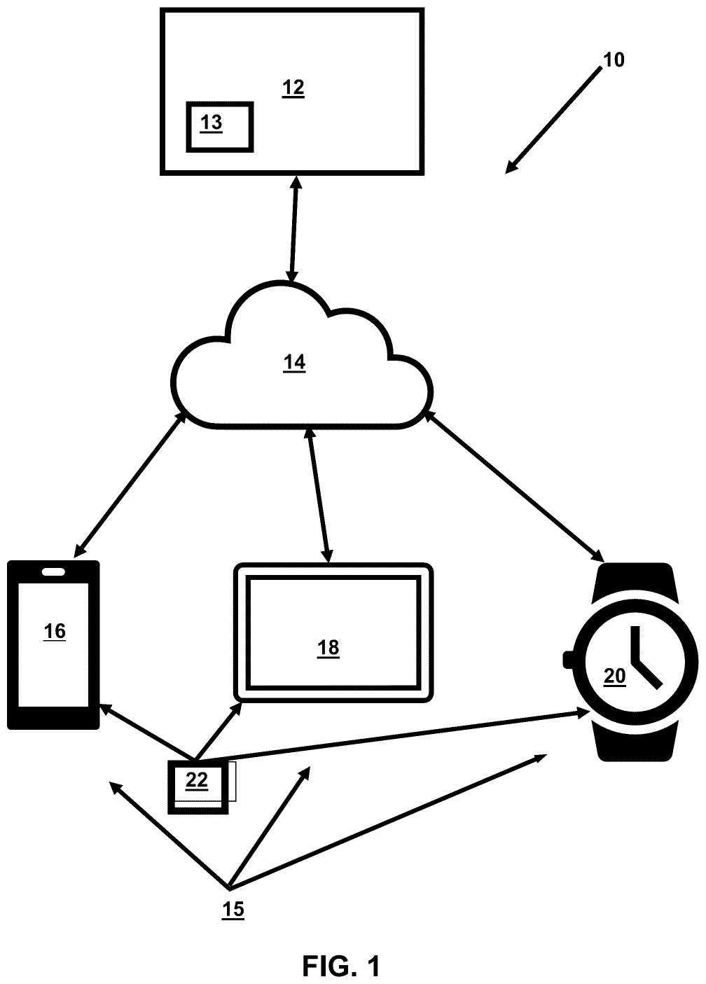

Referring to FIG. 1, a system for dynamic interaction to medical queries propositioned contemporaneously with interaction with medical personnel is generally shown by the numeral 10. A wide variety of wireless devices 15 can provide inputted queries or questions and/or medical data, including a cell phone 16, an electronic tablet 18, and/or a wearable electronic device 20, e.g., a watch. Queries or input can be inputted either through typing or voice recognition. These are merely illustrative examples of the types of wireless devices 15 that can be utilized and should not be deemed limiting in any manner. Optionally, there is at least one wearable sensor 22 that is in electronic communication with the at least one wireless device 15. Sensed health conditions can include cardiovascular activity, e.g., heart rate and heart rate variability, eye health, respiratory and lung health, skin health, daily activity and falling, sleep, car health and cognitive function, and mental health, among other physical conditions.

The wireless devices 16, 18, and/or 20 are in electronic communication with a computer network (hereinafter, “network”) 14. Network 14 can be the Internet, an Internet and/or extranet, or an intranet and/or extranet that is in electronic communication with the Internet. Network 14, in some cases, is a telecommunication and/or data network. Network 14 also can include one or more computer servers, which can enable distributed computing, such as cloud computing. For example, one or more computer servers may enable cloud computing over the network 14 to perform various aspects of analysis, calculation, and generation of the present disclosure, such as acquiring health data from wearable sensors or inputted queries that are either typed or provided through voice recognition,

Network 14 is in electronic communication with a computer system 12, e.g., at least one processor. Computer system 12 can include a central processing unit, processor, or computer processor, which can be a single-core or multi-core processor or a plurality of processors for parallel processing. Computer system 12 also includes memory such as read-only, random-access, and flash memory, among other types of memory. In addition, computer system 12 typically includes an electronic storage unit, a communication interface for communicating with one or more other systems, and peripheral devices such as cache, other memory, data storage, and/or electronic display adapters. The computer system 12 includes at least one processor, which is generally indicated by the numeral 13.

The computer system 12 preferably utilizes a large language model (hereinafter, “LLM”). LLM is a type of artificial intelligence (hereinafter, “AI”) algorithm that uses deep learning techniques and massively large data sets to understand, summarize, generate, and predict new content. LLMs are a type of generative AI specifically architected to help generate text-based content. Language models are commonly used in natural language processing applications where a user inputs a query in natural language to generate a result. An LLM is the evolution of the language model concept in AI that dramatically expands the data used for training and inference. In turn, it provides a massive increase in the capabilities of the AI model. LLM can typically have at least one billion or more parameters, which are the variables present in the model on which it was trained that can be used to infer new content. At their core, large language models, or LLMs, are a type of AI that can mimic human intelligence. Although they use statistical models to analyze vast amounts of data, learning the patterns and connections between words and phrases, an LLM model can change autonomously as it learns. LLMs are trained with immense amounts of data and use self-supervised learning to predict the next token in a sentence, given the surrounding context. The process is repeated over and over until the model reaches an acceptable level of accuracy. An illustrative, but nonlimiting, example of an LLM is ChatGPT.

When queries are inputted through typing or voice recognition by the patient through the wireless devices 16, 18, and/or 20, the computer system, e.g., processor(s) 12 utilizing an LLM can respond and further provide questions requiring input that provides tremendous insight into the medical condition at issue to provide the patient during an examination with all of the requisite information needed for the decisions even if the doctor is not providing all of the necessary information or not providing information in a form that is understandable by the patient. In addition, this inputted data can be optionally augmented by data acquired by at least one wearable sensor 22 on the patient.

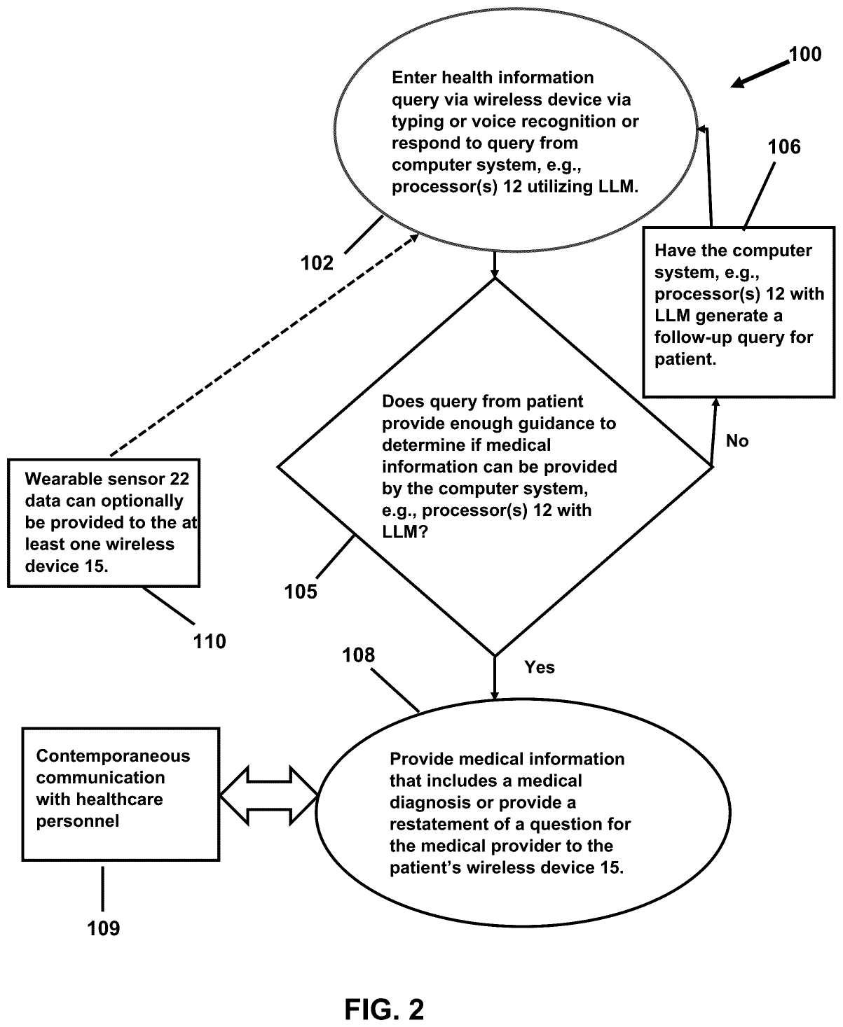

A flowchart of the process can be found in FIG. 2 and is generally indicated by the numeral 100 and is executed with steps indicated by <nnn>. For example, the first step <102> is to enter a health information query via wireless device 15 via typing or voice recognition or respond to a query from a computer system, e.g., processor(s) 12 utilizing LLM. There is then a query as to whether the patient provided enough guidance to determine if the computer system, e.g., processor(s) 12 can provide medical information with LLM <105>. Medical information can include, but is not limited to, a medical diagnosis or a restatement of a query for medical personnel. If the analysis provides a negative determination, then the computer system, e.g., processor(s) 12 proceeds to step <106>, where the computer system, e.g., processor(s) 12 with LLM generates a follow-up query for the patient. The process then returns back to step <102>, where a response by the patient to this query is made. On the other hand, if the analysis in step <105> is a positive determination, then the process proceeds to step <108>, where medical information is provided to a patient's wireless device 15 while simultaneously engaging in contemporaneous communication with healthcare personnel or a restatement of a question or query can be provided that the patient can ask of healthcare personnel <109>.

Optionally, data from the wearable sensor 22 <110> can be provided to the at least one wireless device 15 to provide additional input to the computer system, e.g., processor(s) 12 utilizing LLM to provide additional input in step <102> to facilitate the provision of a medical diagnosis or provide a restatement of a query for medical personnel in step <108> when in contemporaneous communication with the healthcare personnel.

Illustrative, but nonlimiting, examples of wearable sensors 22 include: a heart sensor; an eye sensor; a respiratory sensor; a skin sensor; an activity sensor; a sleep sensor; an ear health sensor; a cognitive function sensor; a mental health sensor; a flexible electronic skin based on a piezoelectric biosensor matrix for monitoring lactic acid, glucose, uric acid, and urea; an intelligent pulse oxygen sensor patch that outputs information such as the blood oxygen, heart rate, and heart rate variability; a one-touch-activated blood diagnostic system for monitoring blood glucose and cholesterol; a skin-like patch for noninvasive blood glucose monitoring; a contact lens sensing device for monitoring blood sugar; a contact lens sensor capable of continuous intraocular pressure monitoring; a smart bandage for optical monitoring of pH; an intelligent bandage for monitoring the uric acid index in a wound; and an ionic bioelectronic skin patch for monitoring of temperature, pressure, pH, and electrocardiogram.

From the preceding, it can be seen that the present disclosure accomplishes at least all of the stated objectives.

### LIST OF REFERENCE CHARACTERS

The following table of reference characters and descriptors are neither exhaustive nor limiting and includes reasonable equivalents. If possible, elements identified by a reference character below and/or those elements which are near ubiquitous within the art can replace or supplement any element identified by another reference character.

TABLE 1
List of Reference Characters

| | |
|---|---|
| 10 | System for dynamic interaction to medical queries propositioned |
| | contemporaneously with interaction with medical personnel. |
| 13 | At least one processor |
| 12 | Computer system, e.g., at least one processor |
| 14 | Computer network |
| 15 | Wireless device |
| 16 | Cell phone |
| 18 | Electronic tablet |
| 20 | Wearable electronic device |
| 22 | Wearable sensor |
| 100 | Flowchart for process steps associated with a system for dynamic |
| | interaction to medical queries propositioned contemporaneously with |
| | interaction with medical personnel. |
| <102> | Process step of entering health information query via wireless device 15 |
| | via typing or voice recognition or responding to a query from a |
| | computer system, e.g., processor(s) 12 utilizing LLM. |
| <105> | Determining if the query from a patient can provide enough guidance to |
| | determine if the computer system, e.g., processor(s) 12 can provide |
| | medical information with LLM. |
| <106> | If there is not enough information in step <105> to provide medical |
| | information, then the computer system, e.g., processor(s) 12 provides a |
| | query to the patent's wireless device 15. |
| <108> | If there is enough information in step <105> to provide a medical |
| | diagnosis or restatement of a query to healthcare personnel, then the |
| | computer system, e.g., processor(s) 12 provides medical information |
| | that includes, but is not limited to, a medical diagnosis or a restatement |
| | of a query for medical personnel, to the patent's wireless device 15. |
| <109> | If there is enough medical information provided in step <102> where |
| | the computer system, e.g., processor(s) 12 with LLM can provide |
| | medical information in step <108>, then there is contemporaneous |
| | medical communication with medical personnel. |
| <110> | Optional data from wearable sensor 22 can be combined with the input |
| | from the wireless device 15 to create medical information for output on |
| | the wireless device utilizing the computer system, e.g., processor(s) 12 |
| | with LLM. Medical information that includes, but is not limited to, a |
| | medical diagnosis or a restatement of a query for medical personnel. |

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A system for dynamic interaction to medical queries propositioned contemporaneously with interaction with medical personnel, the system comprising:
at least one wireless device for inputting at least one query regarding a medical condition;
a computer network in electronic communication with the at least one wireless device; and
at least one processor that utilizes a large language model (LLM) that is in electronic communication with the computer network for analyzing the at least one query from the at least one wireless device and provide at least one follow-up query to the at least one wireless device to generate at least one additional input from the at least one wireless device to provide medical information contemporaneously with communication with medical personnel.

2. The system for dynamic interaction to medical queries propositioned contemporaneously with interaction with medical personnel according to claim 1, wherein the at least one wireless device and the at least one processor that utilizes a large language model (LLM) exchange a plurality of queries through the computer network prior to the at least one processor providing the medical information that is a medical diagnosis.

3. The system for dynamic interaction to medical queries propositioned contemporaneously with interaction with medical personnel according to claim 1, wherein the at least one wireless device and the at least one processor that utilizes a large language model (LLM) exchange a plurality of queries through the computer network prior to the at least one processor providing the medical information that is a restatement of a question for the medical personnel.

4. The system for dynamic interaction to medical queries propositioned contemporaneously with interaction with medical personnel according to claim 1, wherein the at least one wireless device is selected from the group consisting of at least one cell phone, at least one electronic tablet, and at least one wearable electronic device.

5. The system for dynamic interaction to medical queries propositioned contemporaneously with interaction with medical personnel according to claim 1, further includes at least one wearable sensor by a patient for providing additional medical data that is in electronic communication with the at least one wireless device.

6. The system for dynamic interaction to medical queries propositioned contemporaneously with interaction with medical personnel according to claim 5, wherein the wearable sensor is selected from the group consisting of a heart sensor, an eye sensor, a respiratory sensor, a skin sensor, an activity sensor, a sleep sensor, an ear health sensor, a cognitive function sensor, and a mental health sensor.

7. A system for dynamic interaction to medical queries propositioned contemporaneously with interaction with medical personnel, the system comprising:
at least one wireless device for inputting at least one query regarding a medical condition;
a computer network in electronic communication with the at least one wireless device;
at least one wearable sensor by a patient for providing additional medical data that is in electronic communication with the at least one wireless device; and
at least one processor that utilizes a large language model (LLM) that is in electronic communication with the computer network for analyzing the at least one query from the at least one wireless device and provide at least one follow-up query to the at least one wireless device to generate at least one additional input from the at least one wireless device combined with additional medical data from the at least one wearable sensor to provide medical information contemporaneously with communication with medical personnel.

8. The system for dynamic interaction to medical queries propositioned contemporaneously with interaction with medical personnel according to claim 7, wherein the medical information is selected from the group consisting of a medical diagnosis and a restatement of a question for the medical personnel.

9. The system for dynamic interaction to medical queries propositioned contemporaneously with interaction with medical personnel according to claim 7, wherein the at least one wireless device is selected from the group consisting of at least one cell phone, at least one electronic tablet, and at least one wearable electronic device.

10. The system for dynamic interaction to medical queries propositioned contemporaneously with interaction with medical personnel according to claim 7, wherein the wearable sensor is selected from the group consisting of a heart sensor, an eye sensor, a respiratory sensor, a skin sensor, an activity sensor, a sleep sensor, an ear health sensor, a cognitive function sensor, and a mental health sensor.

11. A process for dynamic interaction to medical queries propositioned contemporaneously with interaction with medical personnel, the process comprising:
inputting a query into at least one wireless device regarding a medical condition, wherein the at least one wireless device is in electronic communication with a computer network, wherein the computer network is in electronic communication with at least one processor that utilizes a large language model (LLM);
analyzing the at least one query from the at least one wireless device with the least one processor that utilizes a large language model (LLM);
providing at least one follow-up query to the at least one wireless device from the least one processor that utilizes a large language model (LLM);
receiving at least one additional input from the at least one wireless device; and
providing medical information to the at least one wireless device from the at least one processor that utilizes a large language model (LLM) contemporaneously with interaction with medical personnel.

12. The process for dynamic interaction to medical queries propositioned contemporaneously with interaction with medical personnel according to claim 11, further comprising inputting a plurality of queries with the at least one wireless device with each query analyzed by the at least one processor that utilizes a large language model (LLM) to request an additional input from the at least one wireless device for each query.

13. The process for dynamic interaction to medical queries propositioned contemporaneously with interaction with medical personnel according to claim 11, wherein the at least one wireless device includes at least one cell phone.

14. The process for dynamic interaction to medical queries propositioned contemporaneously with interaction with medical personnel according to claim 11, wherein the at least one wireless device includes at least one electronic tablet.

15. The process for dynamic interaction to medical queries propositioned contemporaneously with interaction with medical personnel according to claim 11, wherein the at least one wireless device includes at least one wearable sensor in electronic communication that obtains additional medical data through at least one wearable sensor by a patient that is in electronic communication with the at least one wireless device.

16. The process for dynamic interaction to medical queries propositioned contemporaneously with interaction with medical personnel according to claim 15, wherein the additional medical data is from the wearable sensor selected from the group consisting of a heart sensor, an eye sensor, a respiratory sensor, a skin sensor, an activity sensor, a sleep sensor, an ear health sensor, a cognitive function sensor, and a mental health sensor.

17. The process for dynamic interaction to medical queries propositioned contemporaneously with interaction with medical personnel according to claim 15, further includes the step of combining the additional medical data is from the wearable sensor with the inputted data regarding a medical condition to obtain a medical diagnosis with the at least one processor that utilizes a large language model (LLM) contemporaneously with interaction with medical personnel.

18. The process for dynamic interaction to medical queries propositioned contemporaneously with interaction with medical personnel according to claim 11, wherein providing medical information to the at least one wireless device includes providing a medical diagnosis.

19. The process for dynamic interaction to medical queries propositioned contemporaneously with interaction with medical personnel according to claim 11, wherein providing medical information to the at least one wireless device includes providing a restatement of a question for the medical personnel.

20. The process for dynamic interaction to medical queries propositioned contemporaneously with interaction with medical personnel according to claim 11, wherein the step of inputting a query into at least one wireless device is selected from the group consisting of a patient typing input and voice recognition of a patient.
